(19) United States
(12) Patent Application Publication
(10) Pub. No.: US 2026/0090773 A1
(43) Pub. Date: Apr. 2, 2026

(54) DYNAMIC TABLE DRIVE ADJUSTMENT SYSTEM FOR MEDICAL IMAGING SYSTEM

(71) Applicant: GE Precision Healthcare LLC, Waukesha, WI (US)

(72) Inventors: Chelsey Amanda Lewis, Waukesha, WI (US); Michelle DeLong Samalik, Franklin, WI (US); John Londt, Oconomowoc, WI (US)

(21) Appl. No.: 18/901,511

(22) Filed: Sep. 30, 2024

(51) Int. Cl. A61B 6/04; A61B 6/00; A61B 6/03; A61B 6/10; A61B 6/46

(52) U.S. Cl. CPC A61B 6/0487; A61B 6/032; A61B 6/0407; A61B 6/102; A61B 6/4435; A61B 6/469

(57) ABSTRACT

An apparatus and method for dynamically adjusting a table drive in a medical imaging system are described. An example apparatus includes a table, a table motor controller to move the table, and a detection system to detect a wall, accessory or patient parameter. The detection system includes at least one of a QR code reader, an RFID reader, an NFC reader, a LiDAR sensor, or a camera. A processing system calculates a table drive set point based on the detected accessory or patient parameter and adjusts the table drive and height to prevent collisions. The apparatus also includes a user interface configured to receive user input of room dimensions, which are a factor in determining at least one of an initial table drive set point and the new table drive set point, and a display device configured to provide a graphical depiction of a new maximum scannable range.

### BACKGROUND

Medical imaging systems, such as computed tomography (CT) imaging systems, often include a table that moves a patient or an accessory through a bore of the imaging system. The table drive mechanism, which controls the movement of the table, is typically set to a fixed length (e.g., a table drive) to prevent collisions with the walls of the imaging room or other objects, including the edges of the bore. This fixed length can restrict the maximum scannable range, thereby limiting the capabilities of the imaging system.

In current systems, there is no mechanism to dynamically adjust the table drive based on the specific dimensions or characteristics of the imaging room, accessories or patients placed on the table. This can lead to inefficiencies, such as the need for multiple adjustments and repositioning, which can disrupt workflow and increase the time required for imaging procedures.

### SUMMARY

This summary introduces concepts that are described in more detail in the detailed description. It should not be used to identify essential features of the claimed subject matter, nor to limit the scope of the claimed subject matter. The present disclosure provides an apparatus and method for dynamically adjusting a table drive in a medical imaging system to enhance workflow efficiency, and expand the maximum scannable range.

In one aspect, an apparatus for dynamically adjusting a table drive in a medical imaging system is provided. The apparatus comprises a table, a table motor drive configured to move the table, and a detection system configured to detect an accessory or patient parameter. The detection system includes at least one of a QR code reader, an RFID reader, an NFC reader, a LiDAR sensor, or a camera. A processing system or computing device is configured to calculate a new table drive set point based on the detected accessory or patient parameter and adjust the table drive and height to prevent collisions with a bore or other objects. The apparatus also includes a user interface configured to receive user input of room dimensions, which are a factor in determining at least one of an initial table drive set point and the new table drive set point, and a display device configured to provide a graphical depiction of a new maximum scannable range.

In another aspect, a method for dynamically adjusting a table drive in a medical imaging system is provided. The method comprises detecting an accessory or patient parameter using a detection system, calculating a new control based on the detected accessory or patient parameter, and adjusting the table drive and height to prevent collisions with a bore or other objects. The method further includes providing a graphical depiction of a new maximum scannable range on a display device and allowing user input of actual room dimensions to enable dynamic adjustments of the table drive and height.

### BRIEF DESCRIPTION OF DRAWINGS

These and other features, aspects, and advantages of the present disclosed subject matter will become better understood when the following detailed description is read with reference to the accompanying drawings in which like characters represent like parts throughout the drawings.

FIG. 1 is a pictorial representation of a CT imaging system, in accordance with aspects of the present disclosure.

FIG. 2 is a block diagram of the CT imaging system in FIG. 1, in accordance with aspects of the present disclosure.

FIG. 3 is a block diagram of an example detection system used with the example CT system of FIGS. 1 and 2.

FIG. 4 is a pictorial representation of a camera of the detection system coupled to the CT system, in accordance with aspects of the present disclosure.

FIG. 5 is another pictorial representation of a camera of the detection system coupled to the CT system, in accordance with aspects of the present disclosure.

FIG. 6 is a pictorial representation of a maximum scannable range of a patient on a table of the example CT system.

FIG. 7 is pictorial representation of a maximum scannable range of an accessory coupled to the table of the example CT system.

FIG. 8 is a flowchart depicting a method for adjusting a maximum scannable range of the example CT system, in accordance with the aspects of the present disclosure.

### DETAILED DESCRIPTION

Embodiments of the present disclosure will now be described, by way of example, with reference to the Figures, in which various embodiments of a medical imaging system and a table for use with a medical imaging system are described. Some imaging systems, such as computed tomography (CT) systems or photon counting computed tomography (PCCT) systems may include a table movable through a bore of the medical imaging system. In some examples, the range of movement of the table may be limited based on external limiting factors, such as the placement of the medical imaging system in the room. For example, the example medical imaging system may be placed in a larger room at some locations than others. Systems placed in larger rooms are able to have an increased range of movement for the table, thereby also increasing a scan range. Further, the maximum scannable range may be set based on potential accessories that could be attached to the table. For example, a scanner placed in a room larger than the minimum specifications for room size may be used with larger or longer accessories to accommodate, for example, taller patients.

The following description relates to an example imaging system including a detection system for detecting one or more objects in the path of the cradle or movable portion of the table and/or one or more accessories attached to the table or cradle, and adjusting the maximum table range, and therefore the maximum scannable range, for each scan by the medical imaging system. Thus, the maxim scannable range can be dynamically updated for each use of the medical imaging system. In the examples described herein, the detection system includes a camera and/or sensors. The camera may help determine a location of objects in the room (e.g., other medical equipment) and or the room constraints (e.g., walls) which would affect the range of movement of the table. The senor may detect accessories attached to the table which may affect the range of movement of the table. Additionally, the camera and/or the sensors may also detect patient parameters (e.g., a height, width, presence of blankets or other stabilizing objects, etc.) that would require adjustment of the range of movement of the table. The maximum range of movement of the table may be adjusted based on the data collected by the camera and/or sensor(s).

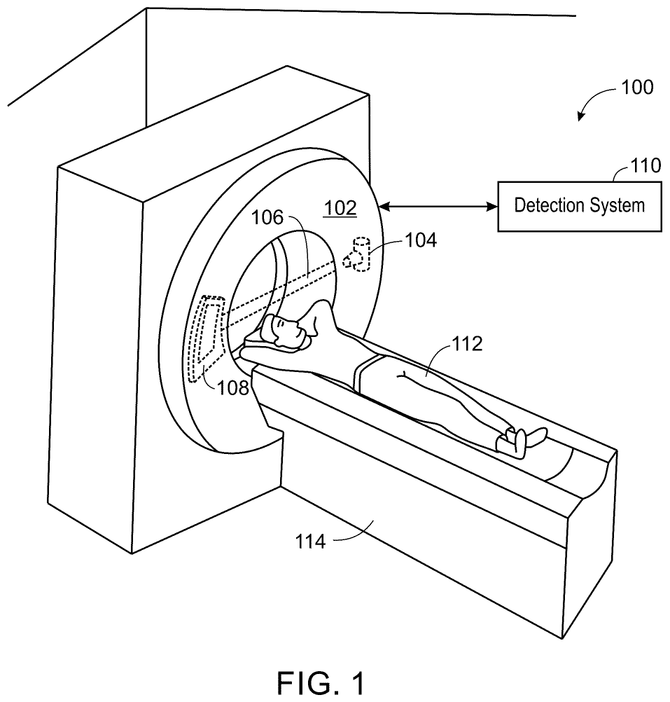

Turning to the figures, FIG. 1 illustrates an exemplary CT system 100. In one example, the CT system 100 may be a PCCT system. Particularly, the CT system 100 is configured to image a subject 112 such as a patient, an inanimate object, one or more manufactured parts, and/or foreign objects such as dental implants, stents, and/or contrast agents present within the body or subject placed on a table 114. The table 114 may be motorized and may be selectively moveable. In one embodiment, the CT system 100 includes a gantry 102, which in turn, may further include at least one X-ray radiation source 104 configured to project an X-ray beam 106 for use in imaging the subject 112. The X-ray radiation source 104 includes an X-ray tube and a target. The X-ray tube generates X-rays by accelerating and focusing a high-energy beam of electrons onto a rotating target. As individual electrons strike the target, the energy released by interacting with the atoms of the target produces X-ray photons isotropically under a polychromatic spectrum, a maximum energy of the X-ray photons matching that of the incident electrons. The X-ray photons leave the tube through a window that defines an X-ray beam. The beam can then be collimated and conditioned using collimator blades and filter(s).

Specifically, the radiation source 104 is configured to project the X-rays 106 towards a detector array 108 positioned on the opposite side of the gantry 102. Although FIG. 1 depicts a single radiation source 104, in certain embodiments, multiple radiation sources may be employed to project a plurality of X-rays 106 for acquiring projection data corresponding to the subject 112 at different energy levels. The radiation source may include an X-ray target manufactured of graphite and metal.

In certain embodiments, the CT system 100 further includes a detection system 110 configured to detect an accessory coupled to the table or a patient parameter. For example, the detection system 110 may use at least one of a QR code reader, an RFID reader, an NFC reader, a LiDAR sensor, or a camera to determine that an accessory is coupled to the table, and determine which accessory is coupled to the table 114. Additionally or alternatively, the detection system 110 may identify a patient parameter (e.g., a size of the patient) that would require an adjustment to a maximum table drive. For example, an exceptionally tall or long patient may require a longer table extender that extends from one end of the table 114, requiring that the maximum table drive be adjusted to prevent collision of the patient and structure of the CT system 100 and/or room in which the CT system 100 is located. Adjusting the maximum table drive prevents movement of the table further than the set maximum table drive, thereby preventing that collision.

The example detection system may be coupled to the CT system (e.g., coupled to a gantry). In such examples, one or more components (e.g., a frame, a bracket, etc.) may be used to couple the detection system to the CT system. Alternatively or additionally, the detection system is coupled to an object adjacent to the CT system, including one or more walls of a room in which the CT system is located, a frame or mount separate from the CT system, etc. In some examples, the detection system may be in multiple separate components positioned apart from one another. For example, a camera may be positioned in a first location and a sensor may be positioned in a second location.

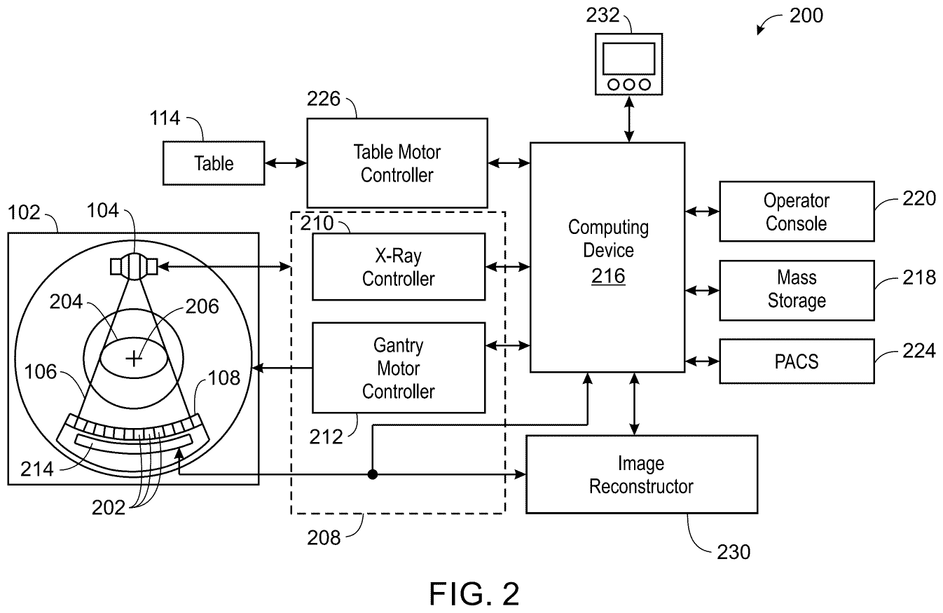

FIG. 2 illustrates an exemplary imaging system 200 similar to the CT system 100 of FIG. 1. In accordance with aspects of the present disclosure, the imaging system 200 is configured for imaging a subject 204 (e.g., the subject 112 of FIG. 1). In one embodiment, the imaging system 200 includes the detector array 108 (see FIG. 1). The detector array 108 further includes a plurality of detector elements 202 that together sense the X-ray radiation beam 106 (see FIG. 2) that pass through the subject 204 (such as a patient) to acquire corresponding projection data. Accordingly, in one embodiment, the detector array 108 is fabricated in a multi-slice configuration including the plurality of rows of cells or detector elements 202. In such a configuration, one or more additional rows of the detector elements 202 are arranged in a parallel configuration for acquiring the projection data.

In certain embodiments, the imaging system 200 is configured to traverse different angular positions around the subject 204 for acquiring desired projection data. Accordingly, the gantry 102 and the components mounted thereon may be configured to rotate about a center of rotation 206 for acquiring the projection data, for example, at different energy levels. Alternatively, in embodiments where a projection angle relative to the subject 204 varies as a function of time, the mounted components may be configured to move along a general curve rather than along a segment of a circle.

As the X-ray source 104 and the detector array 108 rotate, the detector array 108 collects data of the attenuated X-ray beams. The data collected by the detector array 108 undergoes pre-processing and calibration to condition the data to represent the line integrals of the attenuation coefficients of the scanned subject 204. The processed data are commonly called projections.

In some examples, the individual detectors or detector elements 202 of the detector array 108 may include photon-counting detectors which register the interactions of individual photons into one or more energy bins. It should be appreciated that the methods described herein may also be implemented with energy-integrating detectors.

The acquired sets of projection data may be used for basis material decomposition (BMD). During BMD, the measured projections are converted to a set of material-density projections. The material-density projections may be reconstructed to form a pair or a set of material-density map or image of each respective basis material, such as bone, soft tissue, and/or contrast agent maps. The density maps or images may be, in turn, associated to form a volume rendering of the basis material, for example, bone, soft tissue, and/or contrast agent, in the imaged volume.

Once reconstructed, the basis material image produced by the imaging system 200 reveals internal features of the subject 204, expressed in the densities of two basis materials. The density image may be displayed to show these features. In traditional approaches to diagnosis of medical conditions, such as disease states, and more generally of medical events, a radiologist or physician would consider a hard copy or display of the density image to discern characteristic features of interest. Such features might include lesions, vessels, sizes and shapes of particular anatomies or organs, and other features that would be discernable in the image based upon the skill and knowledge of the individual practitioner.

In one embodiment, the imaging system 200 includes a control mechanism 208 to control movement of the components such as rotation of the gantry 102 and the operation of the X-ray source 104. In certain embodiments, the control mechanism 208 further includes an X-ray controller 210 configured to provide power and timing signals to the X-ray source 104. Additionally, the control mechanism 208 includes a gantry motor controller 212 configured to control a rotational speed and/or position of the gantry 102 based on imaging requirements.

In certain embodiments, the control mechanism 208 further includes a data acquisition system (DAS) 214 configured to sample analog data received from the detector elements 202 and convert the analog data to digital signals for subsequent processing. The DAS 214 may be further configured to selectively aggregate analog data from a subset of the detector elements 202 into so-called macro-detectors, as described further herein. The data sampled and digitized by the DAS 214 is transmitted to a computer, processing system, or computing device 216. In one example, the processing system or computing device 216 stores the data in a storage device 218. The storage device 218, for example, may include a hard disk drive, a floppy disk drive, a compact disk-read/write (CD-R/W) drive, a Digital Versatile Disc (DVD) drive, a flash drive, and/or a solid-state storage drive.

Additionally, the processing system or computing device 216 provides commands and parameters to one or more of the DAS 214, the X-ray controller 210, and the gantry motor controller 212 for controlling system operations such as data acquisition and/or processing. In certain embodiments, the computing device or processing system 216 controls system operations based on operator input. The computing device or processing system 216 receives the operator input, for example, including commands and/or scanning parameters via an operator console 220 operatively coupled to the computing device or processing system 216. The operator console 220 may include a keyboard (not shown), a mouse, and/or a touchscreen to allow the operator to specify the commands and/or scanning parameters.

Although FIG. 2 illustrates only one operator console 220, more than one operator console may be coupled to the imaging system 200, for example, for inputting or outputting system parameters, requesting examinations, plotting data, and/or viewing images. Further, in certain embodiments, the imaging system 200 may be coupled to multiple displays, printers, workstations, and/or similar devices located either locally or remotely, for example, within an institution or hospital, or in an entirely different location via one or more configurable wired and/or wireless networks such as the Internet and/or virtual private networks, wireless telephone networks, wireless local area networks, wired local area networks, wireless wide area networks, wired wide area networks, etc.

In one embodiment, for example, the imaging system 200 either includes, or is coupled to, a picture archiving and communications system (PACS) 224. In an exemplary implementation, the PACS 224 is further coupled to a remote system such as a radiology department information system, hospital information system, and/or to an internal or external network (not shown) to allow operators at different locations to supply commands and parameters and/or gain access to the image data.

The computing device or processing system 216 uses the operator-supplied and/or system-defined commands and parameters to operate a table motor controller 226, which in turn, may control a table 114 which may be a motorized table. Specifically, the table motor controller 226 may move the table 114 for appropriately positioning the subject 204 in the gantry 102 for acquiring projection data corresponding to the target volume of the subject 204.

As previously noted, the DAS 214 samples and digitizes the projection data acquired by the detector elements 202. Subsequently, an image reconstructor 230 uses the sampled and digitized X-ray data to perform high-speed reconstruction. Although FIG. 2 illustrates the image reconstructor 230 as a separate entity, in certain embodiments, the image reconstructor 230 may form part of the computing device or processing system 216. Alternatively, the image reconstructor 230 may be absent from the imaging system 200 and instead the computing device or processing system 216 may perform one or more functions of the image reconstructor 230. Moreover, the image reconstructor 230 may be located locally or remotely, and may be operatively connected to the imaging system 200 using a wired or wireless network. Particularly, one exemplary embodiment may use computing resources in a “cloud” network cluster for the image reconstructor 230.

In one embodiment, the image reconstructor 230 stores the images reconstructed in the storage device 218. Alternatively, the image reconstructor 230 may transmit the reconstructed images to the computing device or processing system 216 for generating useful patient information for diagnosis and evaluation. In certain embodiments, the computing device or processing system 216 may transmit the reconstructed images and/or the patient information to a display or display device 232 communicatively coupled to the computing device 216 and/or the image reconstructor 230. In some embodiments, the reconstructed images may be transmitted from the computing device or processing system 216 or the image reconstructor 230 to the storage device 218 for short-term or long-term storage.

The various methods and processes described further herein may be stored as executable instructions in non-transitory memory on a processing system or computing device (or controller) in imaging system 200, such as computing device or processing system 216 and/or image reconstructor 230, where the instructions are executable via one or more processors to carry out the methods and processes described herein. In some embodiments, computing device or processing system 216 may include the instructions in non-transitory memory, and may apply the methods described herein (via one or more processors), at least in part, to projection data (e.g., sinograms) in order to fill in missing views before the projection data is reconstructed via image reconstructor 230. In another embodiment, the methods and processes described herein may be performed by image reconstructor 230. In a still further embodiment, the methods and processes described herein may be distributed across image reconstructor 230 and computing device or processing system 216.

In one embodiment, the display 232 allows the operator to evaluate the imaged anatomy after image reconstruction. The display 232 may also allow the operator to select a volume of interest (VOI) and/or request patient information, for example, via a graphical user interface (GUI) for a subsequent scan or processing, select a scan protocol for carrying out the scan, and the like.

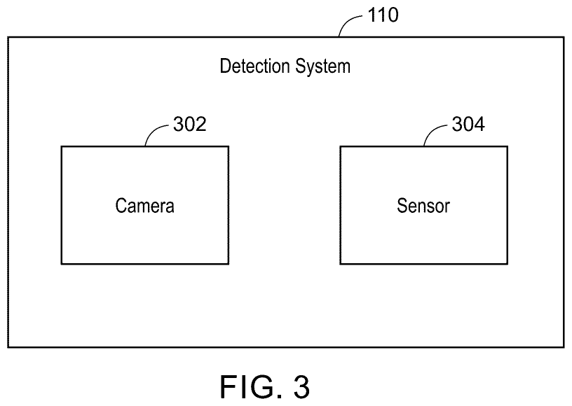

FIG. 3 depicts an example detection system 110 in more detail. The example detection system 110 depicted in FIG. 3 is merely one example detection system 110 and it should be understood that other detection systems may include additional or other components which would allow detection of an accessory or multiple accessories coupled to the table 114 or a patient parameter that would require adjusting the table drive set point, thus increasing a maximum table drive distance. The example detection system 110 is coupled to the CT system 100, and more specifically to the computing device or processing system 216 of the CT system 100. The computing device or processing system 216 receives information or data from the detection system 110 and processes the information to determine how to adjust the maximum table drive (e.g., a maximum range of motion of the table 114). The computing device or processing system 216 uses the information from the detection system 110 to calculate a new table drive set point based on the detected accessory or patient parameter. The computing device may also provide instructions to a table motor controller 226 to adjust the table height and/or table drive in real time, based on the determined table drive set point, to move the table 114 of the CT system 100. In some examples, the detection system 110 continuously or periodically provides data to the computing device or processing system 216, which determines whether the table drive set point needs to be adjusted or updated. The example computing device or processing system 216 is also capable of determining the type of accessory or accessories that may be attached to the table of the CT system 100. The computing device or processing system 216 can use known information (e.g., dimensions) of the type of the accessory to determine the table drive set point. Additionally, the computing device or processing system 216 can determine if the identified accessory is likely to cause a collision (i.e., with a wall, with the bore or the gantry) and provide an alert to the user (i.e., via a display 232, operator console 220, via a speaker, etc.). For example, the identified accessory may extend past the table more than a threshold amount (i.e., a threshold determined during CT system 100 installation and initial table drive set point determination) and will collide with a wall. The determined table drive set point, detected patient parameters, and/or detected accessory may be stored in the mass storage accessible to the computing device or processing system 216 for later access or use by the computing device or processing system 216.

The depicted detection system includes at least one camera 302 (e.g., a 2-D camera, a 3-D camera), and at least one sensor 304. In some examples, the camera 302 is operative to function as a QR reader. In such examples, a user may hold up an accessory having a QR code to the camera 302 to read the QR code to identify the accessory to be attached to the table. The at least one sensor 304 may include an RFID reader, an NFC reader, a LiDAR sensor. In examples including an RFID reader and/or an NFC reader, an RFID or NFC chip or element attached to an accessory can be identified when the accessory is within range of the RFID reader, such as when the accessory is attached to the table 114. In examples using a LiDAR sensor, the LiDAR data is provided to the computing device or processing system 216 of the CT system 100 and the computing device or processing system 216 analyzes the data to determine which accessory is coupled to the table 114. After the accessory coupled to the table 114 is identified, the computing device or processing system 216 determines whether the maximum table drive should be adjusted, and if so, determines the new maximum table drive. In some examples, the detection system 110 includes a proximity sensor coupled to a common collision point (e.g., a wall, a bore of the gantry) as an additional preventative measure to prevent collision of the patient, table 114 and/or the accessory with the wall.

The detection system 110 uses the camera 302 to detect patient parameters (e.g., height) which may require adjustments to the maximum table drive. For example, the data and images from the camera 302 are provided to the computing device or processing system 216 of the CT system 100, which then determines, using the data and images from the camera 302, if the maximum table drive needs to be adjusted. In some examples, the camera 302 is also operative to detect or assist in detecting which accessory or accessories are attached to the table 114 by using the images from the camera 302 to identify the accessory (e.g., based on the same of the accessory, via a QR code, etc.).

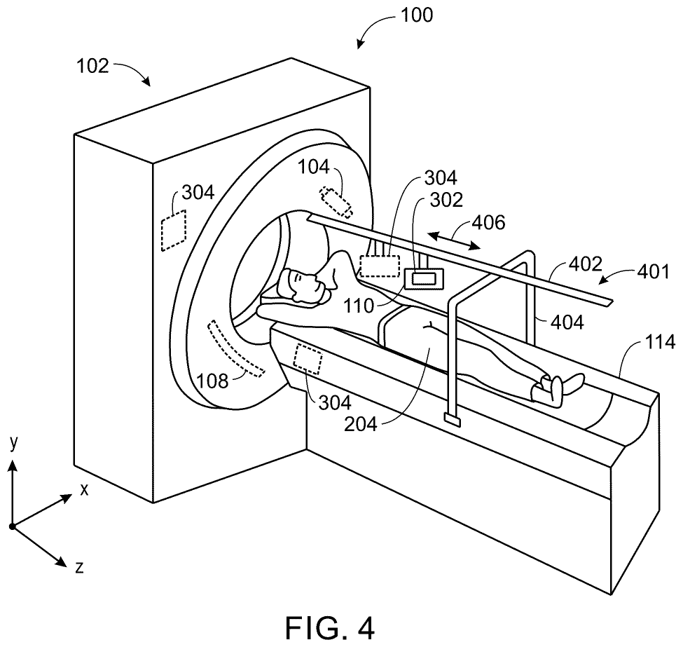

One or more components of the example detection system 110 described herein may be coupled to the CT system 100 using guide rail system 401, such as that depicted in FIG. 4. For example, the detection system 110 or a portion of the detection system 110 (e.g., the camera 302) may be coupled to a guide rail 402 such that the camera 302 is capable of detecting a location of objects in the room (e.g., other medical equipment) and or the room constraints (e.g., walls), patient parameters (e.g., height) and/or identify an accessory is attached to the table 114. In some examples, the room constraints (e.g., dimensions of the room) may be input manually by an operator. In such examples, the room constraints may be input via a user interface on the display at the time of installation of the CT system 100 and may be used to set an initial table drive set point and as a factor in determining adjusted table drive set points. In some examples, a sensor 304 of the detection system 110 is placed adjacent to the camera 302 on the guide rail 402. In some such examples, the camera 302 and sensor 304 are independently movable. In other examples, the sensor 304 of the detection system 110 is located elsewhere. For example, the sensor 304 may be placed directly on the gantry 102 or table 114. Alternatively, the sensor 304 may be located adjacent an operator workstation or console 220, such that the operator my hold an accessory in proximity to the sensor 304 so that the sensor 304 identifies an accessory that is to be attached to the table 114.

FIG. 4 depicts an example CT system 100 with an attached detection system 110. In the illustrated example, a guide rail 402 may be coupled to the CT system 100. The example rail 402 may be coupled to the gantry 102, and may have a support bracket 404 coupled to the table 114. The depicted guide rail 402 and support bracket 404 merely represent an example of how one or more components of the detection system 110 may be coupled to the example CT system 100, and an example rail 402 may be coupled to the CT system 100 using any other suitable means. The example detection system 110 and/or components of the detection system (e.g., a camera 302) that are attached to the guide rail 402 may be movable in at least a first direction 406 along the guide rail 402.

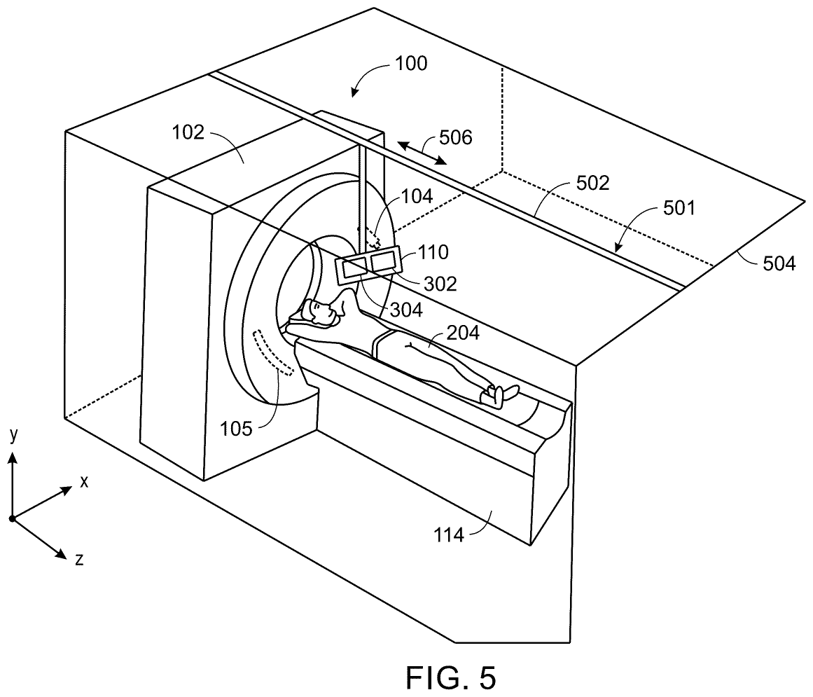

FIG. 5 depicts an example CT system 100 including a detection system 110 coupled to a rail system 501 positioned within the room in which the CT system 100 is located. In the illustrated example, a guide rail 502 is attached to a wall and/or ceiling 504 of a room in which the CT system is located. Similarly to the example in FIG. 4, the example detection system 110 or at least one component of the detection system 110 (e.g., a camera 302, a sensor 304) may be coupled to the guide rail 502. The detection system 110 or at least one component of the detection system 110 may be movable in a first direction 506 along the rail 502. In some examples, both the camera 302 and the sensor 304 of the detection system 110 are coupled to the rail 502. In such examples, the camera 302 and sensor 304 may be coupled to each other and, thus, move with one another. Alternatively, the camera 302 and sensor 304 are independently movable along the rail 502. In some examples, a bi-directional rail system may be coupled to the ceiling and/or walls of the room to further increase the mobility of the detection system 110 or at least component of the detection system 110. In some examples, another component of the detection system 110 (e.g., a sensor 304) may be located elsewhere adjacent to or coupled to the CT system.

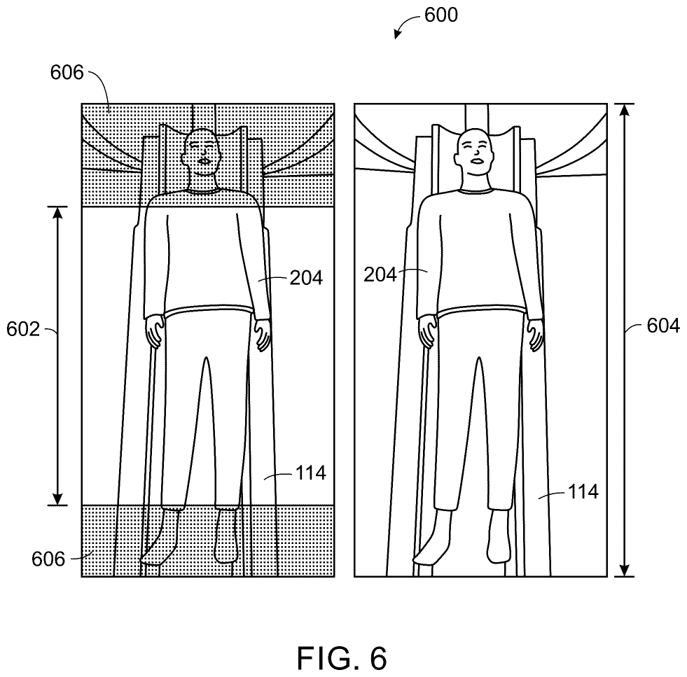

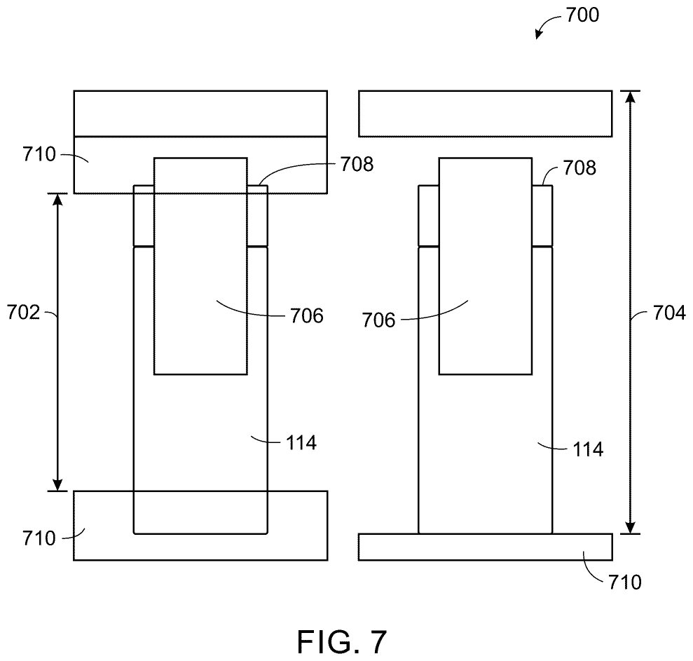

FIGS. 6 and 7 depict example graphical interfaces 600, 700 or portions of graphical user interfaces that may be displayed to the user via the display of a user interface. FIG. 6 depicts an example fixed table drive range 602 or set point that may be currently used with example CT systems 100. As shown, the example fixed table drive range 602 may not allow for the entirety of the patient 204 to be scanned. This fixed table drive range 602 may be set during installation and may be set to account for any accessory that may be attached to the table 114, even if the accessory is not used during every scan. Thus, the maximum scannable range may not be equal to the desired scan range for a patient. Alternatively, FIG. 6 also depicts an example maximum table drive range 604 that may be achieved by setting a new table drive set point that is dynamically updated based on data collected using the detection system 110 described herein. As depicted on the right side of FIG. 6, the maximum table drive range 604 that is achievable when using the detection system 110 to determine the table drive set point enables more of the patient 204 to be scanned, allowing the entirety of the desired scan range to be available during a scan. FIG. 7 similarly depicts a fixed table drive range 702 (left) and a dynamically adjustable table drive range or maximum table drive range 704 (right) in an example where an object 706 (e.g., an accessory, a patient) is extending from a first end 708 of the table 114. As depicted in FIG. 7, the dynamically adjustable drive range 704 allows the table 114 to be moved such that the entirety of the object extending from the table 114 is within the scannable range. Additionally, in some examples, the scan range may be identified using different colored regions 606, 710 to depict what areas are outside of the scan range. The example depictions of FIGS. 6 and 7 may be only a portion of a larger user interface presented to a user via the display.

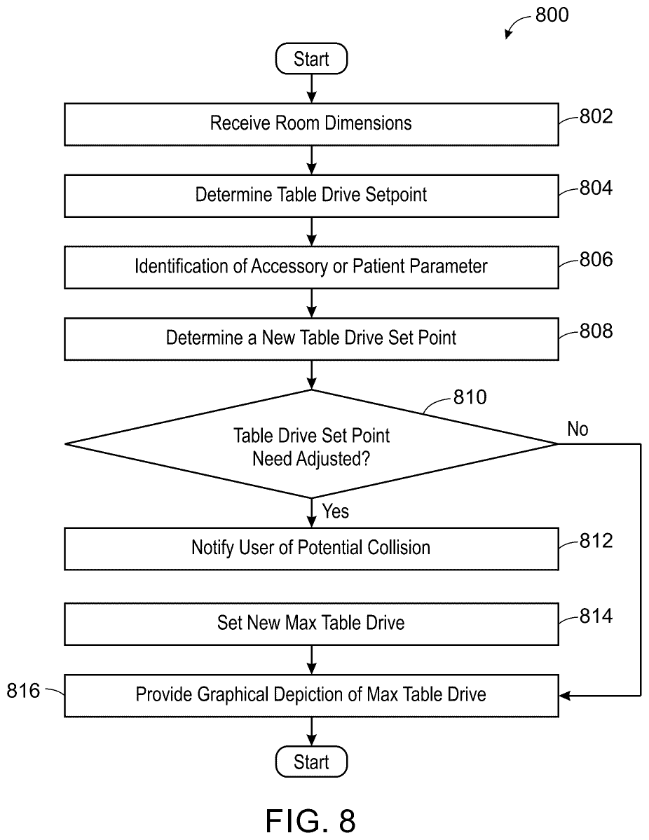

FIG. 8 depicts an example method 800 of adjusting a maximum table drive based on the identified accessory attached to the table 114 or a patient parameter. The method 800 begins with the CT system 100 (e.g., the computing device or processing system 216) receiving room dimensions via input through a user interface (e.g., a display, a keyboard) (block 802). This may be done only during initial installation or setup of a CT system 100, or may be done during a calibration process. Alternatively or additionally, the detection system 110 may be operative to determine the room dimensions and/or distance between a CT system 100 and nearby walls or other objects.

The method 800 continues by determining a table drive set point (e.g., an initial table drive set point) based on the dimensions of the room and the placement of the CT system 100 within the room (block 804). The example computing device or processing system 216 may determine the initial table drive set point and save the initial table drive set point in a memory (e.g., a mass storage) 218 for later user.

The method 800 continues with the identification of one or more accessories attached to the table 114 and/or patient parameters via the detection system 110 (block 806). The detection system 110 may use at least one camera 302 and/or at least one sensor 304 to identify which accessory or accessories are attached to the table 114 and the patient parameters. In the next step, the computing device or processing system 216 determines a new table drive set point or maximum table drive based on the identified accessories or patient parameters (block 808). That is, the computing device or processing system 216 uses the dimensions of the identified accessory to determine the maximum table drive with the accessory attached. Similarly, patient parameters that may affect the maximum table drive may also be detected and be factored into the determination of the new table drive set point. After the new table drive set point is determined, the computing device or processing system 216 determines if the table drive needs to be adjusted (i.e., if the new table drive set point is different from the initial table drive set point or table drive set point currently in use) to provide to the table motor controller 226 to drive the table 114 (block 810). In some examples, the new table drive set point may match the table drive set point currently in use, and thus, there is no need to adjust the table drive. Additionally or alternatively, the new table drive set point may fall within parameters of the current table drive set point, thus the table drive does not need to be adjusted.

If the table drive set point needs to be adjusted, the method 800 continues by notifying the user of a potential collision (block 812) and setting a new maximum table drive by providing the new table drive set point to the table motor controller 226 (block 814). The method 800 concludes by providing a graphical depiction to the user, via a user interface on a display 232 or operator console 220, depicting the new maximum table drive range (block 816). The maximum table drive range may also be displayed to the user if there was no change in the table drive range. The method 800 is complete.

Embodiments of the present disclosure shown in the drawings and described above are example embodiments only and are not intended to limit the scope of the appended claims, including any equivalents as included within the scope of the claims. Various modifications are possible and will be readily apparent to the skilled person in the art. It is intended that any combination of non-mutually exclusive features described herein are within the scope of the present invention. That is, features of the described embodiments can be combined with any appropriate aspect described above and optional features of any one aspect can be combined with any other appropriate aspect. Similarly, features set forth in dependent claims can be combined with non-mutually exclusive features of other dependent claims, particularly where the dependent claims depend on the same independent claim. Single claim dependencies may have been used as practice in some jurisdictions require them, but this should not be taken to mean that the features in the dependent claims are mutually exclusive.

What is claimed is:

1. An imaging system comprising:
a gantry including a source and a detector, and a bore extending through the gantry;
a table;
a table motor coupled to the table and configured to move the table;
a detection system coupled to the imaging system and configured to detect a wall, an accessory or a patient parameter; and
a processing system configured to:
calculate a table drive set point based on the detected wall, accessory, or patient parameter; and
adjust, based on the detected wall, accessory or patient parameter, the table drive and height, via the table motor, to prevent collisions with a bore of the imaging system or other objects adjacent to the imaging system.

2. The imaging system of claim 1, further comprising a display to provide a graphical depiction of a new maximum scannable range via a user interface.

3. The imaging system of claim 2, wherein the user interface configured to receive user input of room dimensions, wherein the room dimensions are a factor in determining at least one of an initial table drive set point and the calculated table drive set point.

4. The imaging system of claim 1, wherein the wherein the processing system is further configured to:
identify the type of accessory placed on the table using the detection system; and
generate an alert if the detected accessory or patient parameter exceeds a predefined threshold that may cause a collision.

5. The imaging system of claim 1, wherein the processing system is further configured to store the detected accessory or patient parameters and the corresponding table drive set points in a database for future reference.

6. The imaging system of claim 1, wherein the processing system is further configured to perform real-time adjustments of the table drive and height based on continuous monitoring of the accessory or patient parameters.

7. The imaging system of claim 1, wherein the detection system is configured to detect multiple accessories simultaneously and adjust the table drive accordingly.

8. A method for dynamically adjusting a table drive in a medical imaging system, comprising:
detecting, using a detection system, a wall, accessory or patient parameter;
calculating a table drive set of a table point based on the detected wall, accessory, or patient parameter; and
adjusting the table drive and height to prevent collisions with a bore or other objects based on the detected wall, accessory, or patient parameter.

9. The method of claim 8, wherein the detection system includes the detection system comprising at least one of a QR code reader, an RFID reader, an NFC reader, a LiDAR sensor, or a camera.

10. The method of claim 9, wherein the camera includes a 3D camera for detecting the height and length of the accessory or patient.

11. The method of claim 8, further comprising receiving user input of room dimensions, wherein the room dimensions are a factor in determining at least one of an initial table drive set point and the calculated table drive set point.

12. The method of claim 11, wherein the initial table drive set point is determined at a time of installation of the medical imaging system.

13. The method of claim 11, further comprising determining a new max scannable range based on the calculated table drive set point.

14. The method of claim 13, further comprising providing a graphical depiction of the new maximum scannable range on a display device.

15. The method of claim 14, wherein the graphical depiction of the new maximum scannable range includes color-coded indicators to identify areas outside the new maximum scannable range.

16. The method of claim 8, further comprising identifying, using the detection system, the type of accessory placed on the table.

17. The method of claim 8, further comprising generating an alert if the detected accessory or patient parameter exceeds a predefined threshold that may cause a collision.

18. The method of claim 8, further comprising storing the detected accessory or patient parameters and the corresponding table drive set points in a database for future reference.

19. The method of claim 8, wherein the adjustment of the table drive and height is performed in real-time based on continuous monitoring of the accessory or patient parameters.

20. The method of claim 8, wherein detecting an accessory includes detecting multiple accessories simultaneously.
